(19) United States
(12) Patent Application Publication
(10) Pub. No.: US 2005/0129638 A1
(43) Pub. Date: Jun. 16, 2005

(54) Make-up composition comprising a particulate material enclosing colored inorganic pigments and inorganic pigments

(75) Inventor: Christophe Dumousseaux, Kawasaki-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL

(21) Appl. No.: 10/982,922

(22) Filed: Nov. 8, 2004

(30) Foreign Application Priority Data
Nov. 7, 2003 (JP) 2003-378103

(51) Int. Cl. A61K 7/021; A61K 7/06; A61K 7/11

(52) U.S. Cl. 424/63; 424/70.13

(57) ABSTRACT

Disclosed herein are a make-up cosmetic composition comprising from 0.5 to 25% by weight of at least one particulate material having a volume average particle size ranging from 1 to 20 μm and enclosing at least one colored inorganic pigment and from 0.2 to 8% by weight of at least one inorganic pigment chosen from white and colored inorganic pigments, and use thereof. This make-up cosmetic composition can provide sufficient hiding power and transparency as well as natural appearance after application on skin.

The present disclosure relates to make-up compositions comprising from 0.5% to 25% by weight of at least one particulate material having a volume average particle size ranging from 1 to 20 μm and enclosing at least one colored inorganic pigment and from 0.2% to 8% by weight of at least one inorganic pigment chosen from white and colored inorganic pigments, wherein the percentages are relative to the total weight of the composition. The cosmetic make-up compositions as disclosed herein can impart good coverage and an excellent feeling of transparency on human skin.

One of the main purposes of a make-up foundation is to provide a smooth and even looking skin tone. This effect is traditionally obtained by adding different kinds of powders to a make-up composition. For example, conventional inorganic pigments, such as titanium oxide and iron oxides, have been used in cosmetic make-up compositions to cover defects on skin such as blots and freckles and to create an even looking effect. However, the use of a high level of these pigments tends to make the appearance very unnatural, or “mask-like.” At the same time, an accumulation of pigments may often be observed in fine lines and wrinkles, and instead of hiding these defects, such conventional make-up compositions tend to highlight them.

In order to solve this problem, transparent fillers of different shapes have been used in combination with inorganic pigments. These fillers can combine a good transparency and a strong diffusion of light. They are helpful in obtaining a good soft focus effect (R. Emmert, Cosmetic&Toiletries, vol.111, no.7, p.57 (1996)). However, when these fillers are used in combination with a high level of inorganic pigments, the transparency of the formulation tends to be very low and the soft focus effect may be completely lost. On the contrary, if no pigments or a very low level of pigments are used, the covering power of the make-up product may not be sufficient to reduce the appearance of skin discolorations.

Attempts have been made to use composite particles that combine pigments and fillers. Pigments can be enclosed and dispersed inside different kinds of fillers, or pigments can be applied on the surface of fillers. However, the use of this type of pigments alone may not give suitable covering power and the use in an inappropriate ratio of traditional pigments may cancel the benefits of these composite particles.

Therefore, there is a great need for a make-up composition that can be very transparent, and at the same time can appear natural and have a high covering power after application on the skin.

The present inventors have surprisingly discovered that, by combining, in an appropriate ratio, at least one particulate material enclosing at least one colored inorganic pigment with at least one inorganic pigment chosen from white and colored inorganic pigments, it is possible to obtain a make-up composition with a satisfactory hiding power and an excellent feeling of transparency.

The make-up composition as disclosed herein comprises from 0.5% to 25% by weight, such as from 1% to 20% by weight, and further such as from 1% to 15% by weight of at least one particulate material enclosing at least one colored inorganic pigment and from 0.2% to 8% by weight, such as from 0.5% to 7% by weight, and further such as from 1% to 6% by weight of at least one inorganic pigment chosen from white and colored inorganic pigments. The percentages are relative to the total weight of the composition.

The at least one particulate material as disclosed herein has a volume average particle size ranging from 1 to 20 μm. For a smaller particle size, i.e., smaller than 1 μm, it would be very difficult to introduce and disperse the pigments homogeneously inside the material. For a larger particle size, i.e., larger than 20 μm, sensorial properties during application and optical properties after application may be decreased. Colored particles with larger particulate size may have a high forward scattering and therefore may provide almost no covering power in the formulation. The volume average particle size (i.e., D50 in volume) can be measured with a powder laser granulometer, either in air or in ethanol medium. Commercial instruments are available from Malvern Instrument, Horiba Ltd. or Coulter companies.

As used herein, the term “enclosing at least one colored inorganic pigment” means that at least one colored inorganic pigment is located inside the particulate material. Therefore, the composite particulate material as disclosed herein does not refer to particles, which are covered or coated by one layer or multilayer of pigments on their surface. In the composite particulate material as disclosed herein, the colored inorganic pigments are well and homogeneously dispersed inside the particulate material. The appropriate choice of the base material and the percentage of the pigments inside can provide a good compromise of optical properties. Processes to obtain this kind of the particulate material are, for example, well described in JP-B-2591946 (Sumitomo Chemical), JP-B2861806 (NSG), JP-A-H08-239310 (NSG), JP-A-H06-47273 (Suzuki Oil and Fat) in the case of inorganic based particulate materials, and in JP-A-2001-354776 (Shinetsu Chemical) in the case of silicone elastomer based particulate materials. An advantage of this type of composite particulate material is the absence of interaction between the enclosed pigments and water or oil. Therefore, there may be no change of color after application of the make-up composition, and/or no dulling effect with time when sweat or sebum starts to wet the composition.

As used herein, the term “colored inorganic pigments” means metal oxide pigments traditionally used to match the skin color in cosmetic compositions. The color of these pigments could be red, yellow, blue, green, violet, brown or black. Examples of the colored inorganic pigments include iron oxide, ultramarine blue, Prussian blue, ferric blue, manganese violet, and chromium oxide. Further examples include mixed or complex pigments comprising a mixture of metal oxides such as mixed titanium-iron oxides. These pigments have, for example, a primary particle size ranging from 0.1 to 1.5 μm. Smaller and larger particle sizes may not be adequate in terms of hiding power. As used herein, the term “primary particle size” means the average particle size of 100 particles, which is determined using electron microscopy, such as Scanning Electron Microscopy or Transmission Electron Microscopy.

The base material of the at least one particulate material as disclosed herein can be organic or inorganic. The organic materials include, for example, cellulose-based polymers, silicone resins, silicone elastomers, acrylic polymers, polyurethane-based polymers, polyamides, polyethylenes, and polystyrenes. The inorganic materials include, for example, glass, silicon dioxide and silicon-based composite oxides.

There is no limitation in terms of shape of the at least one particulate material. It can be, for example, in the shape of a sphere, flake, rod or needle. The particulate material can be porous or nonporous.

The at least one particulate material as disclosed herein has a refractive index of less than 1.8, such as less than 1.6, in order to enhance the transparency after application on skin. In fact, the transparency of the at least one particulate material, when wetted by the oil present in the formulation, tends to be high. The refractive index can readily be determined by one of ordinary skill in the art using known techniques.

The percentage of the colored inorganic pigments inside the particulate material ranges from 3% to 60% by weight relative to the total weight of the particulate material. If the percentage is lower, i.e., lower than 3% by weight, the particulate material may not be able to give enough hiding power. If the percentage is higher, i.e., higher than 60% by weight, it tends to be difficult to satisfactorily synthesize the particulate material. The at least one particulate material as disclosed herein may enclose one type of colored inorganic pigment, a mixture of colored inorganic pigments or a mixture of white and colored inorganic pigments.

Examples of commercially available particulate materials enclosing at least one colored inorganic pigment include products based on polymethylmethacrylate available from Ganz Chemical (Ganzpearl), products based on cellulose available from Daito (Cellulobeads), and products based on porous silica available from Miyoshi (PC Ball).

The inorganic pigments used in combination with the at least one particulate material enclosing at least one colored inorganic pigments may be chosen from white pigments, colored pigments, and a mixture thereof.

As used herein, the term “white inorganic pigments” means white metal. oxide pigments traditionally used in cosmetic compositions, which may be surface treated. Examples include titanium oxide, zinc oxide, cerium oxide and zirconium oxide. These pigments have a primary particle size ranging from 0.1 to 1.5 μm. Smaller and larger particle sizes may not be adequate in terms of hiding power.

The colored inorganic pigments are as described above.

The at least one inorganic pigment may be surface treated with at least one hydrophobic agent in order to improve their dispersibility in the fatty phase. The hydrophobic treatment agent may be chosen from silicones such as methicones, dimethicones, and perfluoroalkylsilanes; fatty acids such as stearic acid; metal soaps such as aluminum dimyristate, and the aluminum salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, and amino acids; N-acylated amino acids and the salts thereof; lecithin, isopropyl triisostearyl titanate, and mixtures thereof.

The N-acylated amino acids may comprise at least one acyl group having from 8 to 22 carbon atoms, chosen, for example, from 2-ethylhexanoyl, caproyl, lauroyl, myristoyl, palmitoyl, stearoyl and cocoyl groups. The salts of these compounds may be chosen from aluminium, magnesium, calcium, zirconium, zinc, sodium and potassium salts. The amino acids may be chosen, for example, from lysine, glutamic acid and alanine.

The term “alkyl” mentioned in the compounds above means, for example, an alkyl group having from 1 to 30 carbon atoms, such as from 5 to 16 carbon atoms.

The composition as disclosed herein may also comprise at least one dyestuff chosen, for example, from pulverulent compounds and liposoluble dyes, for example, in an amount ranging from 0.01% to 50% by weight relative to the total weight of the composition. The pulverulent compounds may be chosen from the organic pigments and nacres usually used in cosmetic compositions. In one embodiment, the pulverulent compounds are present in an amount ranging from 0.1% to 25% by weight, such as from 1% to 20% by weight relative to the total weight of the composition.

Among the organic pigments that may be used in the composition disclosed herein, examples include pigments of D & C type and lakes based on cochineal carmine or on barium, strontium, calcium or aluminum.

The nacreous pigments may be chosen from white nacreous pigments such as mica coated with titanium or with bismuth oxychloride, colored nacreous pigments such as titanium mica with iron oxides, titanium mica with, for example, ferric blue or with chromium oxide, titanium mica with an organic pigment of the abovementioned type, and also nacreous pigments based on bismuth oxychloride.

The composition may also comprise at least one filler, which may be chosen from those that are well known to those skilled in the art and which is commonly used in cosmetic compositions. The fillers may be mineral or organic, and lamellar or spherical. Mention may be made, for example, of talc, mica, silica, kaolin, Nylon powder (Orgasol from Atochem), poly-β-alanine powder, polyethylene powder, Teflon, lauroyllysine, starch, boron nitride, tetrafluoroethylene polymer powders, hollow microspheres such as Expancel (Nobel Industrie), Polytrap (Dow Corning), silicone resin microbeads (for example Tospearls from Toshiba), precipitated calcium carbonate, magnesium carbonate, magnesium hydrocarbonate, hydroxyapatite, hollow silica microspheres (Silica Beads from Maprecos), glass or ceramic microcapsules, and metal soaps derived from organic carboxylic acids comprising from 8 to 22 such as from 12 to 18 carbon atoms, for example, zinc stearate, magnesium stearate, lithium stearate, zinc laurate and magnesium myristate.

The composition may also comprise at least one additive chosen from the additives usually used in cosmetic compositions, such as thickeners, preserving agents, fragrances, sunscreens, free-radical scavengers, waxes, moisturizers, vitamins, proteins, sequestrants, ceramides, acidifying or basifying agents, and emollients.

The composition can comprise at least one volatile oil. The term “volatile oil” means any non-aqueous medium that is capable of evaporating from the skin in less than one hour. The volatile phase in the composition comprises oils with a vapor pressure, at room temperature and atmospheric pressure, ranging from 10⁻² to 300 mmHg (1.33 Pa to 40 000 Pa).

These volatile oils can be chosen from volatile hydrocarbon-based oils and silicone oils optionally comprising at least one group chosen from alkyl and alkoxy groups at the end of the silicone chain and/or pendent on the chain.

As volatile silicone oils which can be used herein, mention may be made, for example, of linear or cyclic silicones comprising from 2 to 7 silicon atoms, wherein these silicones optionally comprise at least one group chosen from alkyl and alkoxy groups comprising from 1 to 10 carbon atoms. Mention may also be made, for example, of octamethylcyclotetrasiloxane, decamethylcyclopentasiloxane, hexadecamethylcyclohexasiloxane, heptamethylhexyltrisiloxane and heptamethyloctyltrisiloxane.

The volatile hydrocarbon-based oils that may be mentioned include, for example, C₈-C₁₆ isoparaffins such as isooctane, isododecane, isodecane, heptane, and isohexadecane and mixtures thereof.

The composition disclosed herein can also comprise at least one non-volatile oil chosen, for example, from non-volatile hydrocarbon-based oils, silicone oils and fluoro oils.

The non-volatile hydrocarbon-based oils which may be used in the composition as disclosed herein include, for example:
hydrocarbon-based plant oils such as triglycerides comprising fatty acid esters and glycerol wherein the fatty acids may have varied chain lengths ranging, for example, from C₄ to C₂₄, these chains possibly being linear or branched, and saturated or unsaturated; these oils are chosen, for example, from wheat germ oil, sunflower oil, grape seed oil, sesame oil, corn oil, apricot oil, castor oil, karite butter, avocado oil, olive oil, soybean oil, sweet almond oil, palm oil, rape seed oil, cotton oil, hazelnut oil, macadamia oil, jojoba oil, alfalfa oil, poppy oil, pumpkin oil, marrow oil, blackcurrant seed oil, evening primrose oil, millet oil, barley oil, quinoa oil, rye oil, safflower oil, candlenut oil, passion flower oil and musk rose oil; and caprylic/capric acid triglycerides such as those sold by Stearineries Dubois and those sold under the names Miglyol 810, 812 and 818 by Dynamit Nobel;
synthetic ethers comprising from 10 to 40 carbon atoms;
linear or branched hydrocarbons of mineral or synthetic origin, such as petroleum jelly, polydecenes, hydrogenated polyisobutene such as parleam, and squalane, and mixtures thereof;
synthetic esters such as oils of formula R₁COOR₂ wherein R₁ is chosen from linear and branched fatty acid residues comprising from 1 to 40 carbon atoms and R₂ is chosen from linear and branched hydrocarbon-based chains comprising from 1 to 40 carbon atoms (in one embodiment, R₂ is chosen from branched hydrocarbon-based chains), provided that the number of the carbon atoms in (R₅+R₆) is larger than 10. Examples include purcellin oil (cetostearyl octanoate), isopropyl myristate, isopropyl palmitate, C₁₂-C₁₅ alkyl benzoate, hexyl laurate, diisopropyl adipate, isononyl isononanoate, 2-ethylhexyl palmitate, isostearyl isostearate, alkyl and polyalkyl octanoates, decanoates and ricinoleates such as propylene glycol dioctanoate; hydroxylated esters such as isostearyl lactate and diisostearyl malate; and pentaerythritol esters;
fatty alcohols that are liquid at room temperature, comprising at least one carbon-based chain chosen from branched and unsaturated carbon-based chains comprising from 12 to 26 carbon atoms, for instance octyidodecanol, isostearyl alcohol, oleyl alcohol, 2-hexyldecanol, 2-butyloctanol and 2-undecylpentadecanol;
higher fatty acids such as oleic acid, linoleic acid and linolenic acid; and mixtures thereof.

The non-volatile silicone oils which may be used in the composition as disclosed herein may be chosen, for example, from non-volatile polydimethylsiloxanes (PDMSs), polydimethylsiloxanes comprising at least one group chosen from alkyl and alkoxy groups that is pendent and/or at the end of a silicone chain, wherein the at least one group comprises from 2 to 24 carbon atoms, and phenylsilicones, for instance phenyltrimethicones, phenyldimethicones, phenyltrimethylsiloxydiphenylsiloxanes, diphenyidimethicones, diphenylmethyldiphenyltrisiloxanes and 2-phenylethyl trimethylsiloxysilicates.

The fluoro oils which may be used in the composition as disclosed herein include, for example, fluorosilicone oils, fluoropolyethers and fluorosilicones, as described in document EP-A-847 752.

The composition can comprise an aqueous phase comprising water. The water may be chosen, for example, from floral water such as cornflower water, mineral water such as VITTEL water, LUCAS water and LA ROCHE POSAY water and thermal water.

The aqueous phase may also comprise at least one solvent other than water, chosen, for example, from primary alcohols such as ethanol and isopropanol, glycols such as propylene glycol, butylene glycol, dipropylene glycol, diethylene glycol, glycol ethers such as (C₁-C₄)alkyl ether of mono-, di- or tripropylene glycol, mono-, di- or triethylene glycol, and mixtures thereof.

The aqueous phase may further comprise at least one stabilizing agent chosen, for example, from sodium chloride, magnesium dichloride and magnesium sulphate.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Following non-limiting examples and comparative examples are given to illustrate the invention. The numbers shown below in the compositions are percentages by weight relative to the total weight of the composition.

### EXAMPLE 1 AND COMPARATIVE EXAMPLES 1 TO 3

Liquid foundations having the following compositions were prepared.

TABLE 1

| | Exam- | Compar- | Compar- | |
|---|---|---|---|---|
| | ple 1 | ative | ative | |
| | (inven- | Exam- | Exam- | Comparative |
| | tive) | ple 1 | ple 2 | Example 3 |
| Phase I | | | | |
| Dimethicone copolyol | 5 | 5 | 5 | 5 |
| Dimethicone | 4 | 4 | 4 | 4 |
| Cyclomethicone | 14.5 | 14.5 | 9.5 | 17.5 |
| Isododecane | 10.5 | 10.5 | 10.5 | 10.5 |
| Bentone gel | 10 | 10 | 10 | 10 |
| Plastic Powder D400¹ | 4 | 8 | 4 | 4 |
| Cellulobeads D-5 (W)² | 6.7 | 6.7 | 6.7 | — |
| Cellulobeads D-5 (Y)² | 1.0 | 1.0 | 1.0 | — |
| Cellulobeads D-5 (R)² | 0.3 | 0.3 | 0.3 | — |
| Amino acid treated | 3.3 | — | 7.4 | 7.4 |
| titanium oxide³ | | | | |
| Amino acid treated iron | 0.7 | — | 1.6 | 1.6 |
| oxide³ | | | | |
| Phase II | | | | |
| Water | 31.2 | 31.2 | 31.2 | 31.2 |
| Butylene glycol | 8 | 8 | 8 | 8 |
| Magnesium sulfate | 0.8 | 0.8 | 0.8 | 0.8 |

¹Manufactured by Toshiki Pigment.
²Cellulobeads are spherical cellulose beads manufactured by Daito Kasei and are enclosing 33% by weight of titanium oxide (W), yellow iron oxide (Y) or red iron oxide (R) pigments respectively. Volume average particle size is 8.3 μm for Cellulobeads D-5 (Y) and 9.9 μm for Cellulobeads D-5 (R). The volume average particle size for Cellulobeads D-5(W) can be determined readily by one of ordinary skill in the art.
³NAI series of titanium oxide and iron oxide available from Miyoshi.

Amino acid treated titanium oxide and amino acid treated iron oxide were premixed with a part of cyclomethicone on a 3 roll mill and added to the rest of Phase I. Phase II was mixed separately, and then added to phase I using a conventional homogenizer.

For these examples of compositions, hiding power and transparency were measured. As used herein, the term “hiding power” means the ability to hide color defects on skin.

Measurement of Hiding Power

Samples were applied on Erichsen hiding power charts (Type 24/5) with a thickness of 30 μm using an applicator and let dry 2 hours at room temperature. The color difference ΔE between the black and the white side of the chart was then measured with a Minolta CR-300 colorimeter in a reflectance mode. The color difference is obtained using Hunter's color difference formula ΔE=[(ΔL)²+(Δa)²+(Δb)²]^{0.5}. The reported value shown below is an average of 9 measurements.

Measurement of Transparency:

Mean value ranging from 400 to 700 nm of the total transmittance was measured using an integrating sphere with a JASCO (Nihon Bunkou) V-550 spectrophotometer. The reported value shown below is an average of 6 measurements. All measurements were made using a SPF quartz cell (20 μm thickness) on a film after drying 10 minutes at 37° C. The results are shown in Table 2.

TABLE 2

| | Example 1 | Comparative | Comparative | Comparative |
|---|---|---|---|---|
| | (inventive) | Example 1 | Example 2 | Example 3 |
| Hiding Power | 27 | 38 | 21 | 25 |
| (ΔE) | | | | |
| Transparency (%) | 39 | 73 | 22 | 27 |

As shown in Table 2, the composition in accordance with the present disclosure shows a very good compromise between hiding power and transparency. Furthermore this composition has a good retention of color with time after application on skin.

### EXAMPLE 2

A powder foundation having the following composition was prepared.

| | Example 2 |
|---|---|
| Talc | 37.85 |
| Sericite | 31.5 |
| Mica | 4.5 |
| Titanium oxide | 4.9 |
| Iron oxide | 1.98 |
| Zinc stearate | 0.9 |
| Liquid paraffin | 3.4 |
| Phenyl Trimethicone | 4.5 |
| Parabens | 0.27 |
| Cellulobeads D-5 (W) | 8.4 |
| Cellulobeads D-5 (Y) | 1.2 |
| Cellulobeads D-5 (R) | 0.4 |

The composition of Example 2 has a good spreadability to skin, gives a natural finish with a good covering effect, makes fine lines and wrinkles hard to be seen and has a good retention of color.

1. A make-up composition, comprising
from 0.5 to 25% by weight of at least one particulate material having a volume average particle size ranging from 1 to 20 μm and enclosing at least one colored inorganic pigment and
from 0.2 to 8% by weight of at least one inorganic pigment chosen from white and colored inorganic pigments,
wherein the percentages are relative to the total weight of the composition.

2. The composition according to claim 1, wherein the composition comprises from 1 to 20% by weight of the at least one particulate material relative to the total weight of the composition.

3. The composition according to claim 2, wherein the composition comprises from 1 to 15% by weight of the at least one particulate material relative to the total weight of the composition.

4. The composition according to claim 1, wherein the composition comprises from 0.5 to 7% by weight of the at least one inorganic pigment relative to the total weight of the composition.

5. The composition according to claim 4, wherein the composition comprises from 1 to 6% by weight of the at least one inorganic pigment relative to the total weight of the composition.

6. The composition according to claim 1, wherein the at least one particulate material comprises at least one base material chosen from cellulose-based polymers, silicone resins, silicone elastomers, acrylic polymers, polyurethane-based polymers, polyamides, polyethylenes, polystyrenes, and silica-based materials.

7. The composition according to claim 6, wherein the silica-based materials are chosen from glass, silicon dioxide and silicon-based composite oxides.

8. The composition according to claim 6, wherein the at least one particulate material comprises at least one base material chosen from cellulose-based polymers and silica-based materials.

9. The composition according to claim 1, wherein the at least one particulate material has a refractive index of less than 1.8.

10. The composition according to claim 1, wherein the at least one colored inorganic pigment enclosed in the at least one particulate material is chosen from iron oxide, ultramarine blue, Prussian blue, ferric blue, manganese violet, chromium oxide, mixed and complex pigments comprising a mixture of metal oxides.

11. The composition according to claim 10, wherein the mixture of metal oxides includes mixed titanium-iron oxides.

12. The composition according to claim 1, wherein the at least one colored inorganic pigment enclosed in the at least one particulate material has a primary particle size ranging from 0.1 to 1.5 μm.

13. The composition according to claim 1, wherein the at least one colored inorganic pigment enclosed in the at least one particulate material is present in an amount ranging from 3 to 60% by weight relative to the total weight of the particulate material.

14. The composition according to claim 1, Wherein the at least one inorganic pigment has a primary particle size ranging from 0.1 to 1.5 μm.

15. The composition according to claim 1, wherein the at least one inorganic pigment is surface treated with at least one hydrophobic agent.

16. The composition according to claim 15, wherein the at least one hydrophobic agent is chosen from silicones, fatty acids, metal soaps, N-acylated amino acids and the salts thereof, lecithin, and isopropyl triisostearyl titanate.

17. The composition according to claim 1, further comprising at least one additional ingredient chosen from at least one dyestuff, at least one filler, at least one volatile oil, and at least one non-volatile oil.

18. The composition according to claim 1, comprising an aqueous phase.

19. The composition according to claim 18, wherein the aqueous phase comprises at least one solvent other than water.

20. The composition according to claim 18, wherein the aqueous phase comprises at least one stablilizing agent.

21. The composition according to claim 1, wherein the composition is a foundation.

22. A cosmetic process for making up skin, comprising
applying to the skin a make-up composition comprising
from 0.5 to 25% by weight of at least one particulate material having a volume average particle size ranging from 1 to 20 μm and enclosing at least one colored inorganic pigment and
from 0.2 to 8% by weight of at least one inorganic pigment chosen from white and colored inorganic pigments,
wherein the percentages are relative to the total weight of the composition.

23. A cosmetic process for balancing the hiding power and the transparency effect of a make-up composition, comprising
applying to the skin a make-up composition comprising
from 0.5 to 25% by weight of at least one particulate material having a volume average particle size ranging from 1 to 20 μm and enclosing at least one colored inorganic pigment and
from 0.2 to 8% by weight of at least one inorganic pigment chosen from white and colored inorganic pigments,
wherein the percentages are relative to the total weight of the composition, and wherein the at least one particulate material and the at least one inorganic pigment are present in an amount effective to allow balance of the hiding power and the feeling of transparency in said make-up composition.

24. A method of manufacturing a make-up composition, comprising
including in the composition from 0.5 to 25% by weight of at least one particulate material having a volume average particle size ranging from 1 to 20 μm and enclosing at least one colored inorganic pigment and
from 0.2 to 8% by weight of at least.one inorganic pigment chosen from white and colored inorganic pigments,
wherein the percentages are relative to the total weight of the composition.
